(19) United States
(12) Patent Application Publication
(10) Pub. No.: US 2022/0211598 A1
(43) Pub. Date: Jul. 7, 2022

(54) DIESTER OF PLANT-BASED 1,3-PROPANEDIOL

(71) Applicant: SYMRISE AG, Holzminden (DE)

(72) Inventors: Martina KÖNIG, Hamburg (DE); Martina ISSLEIB, Hoisdorf (DE); Lars JUNG, Hamburg (DE)

(21) Appl. No.: 17/423,331

(22) PCT Filed: Jan. 15, 2019
(86) PCT No.: PCT/EP2019/050953

§ 371 Date: Jul. 15, 2021

(51) Int. Cl. A61K 8/37; C07C 69/28; A61Q 17/04; A61Q 19/00

(52) U.S. Cl. CPC A61K 8/375; C07C 69/28; A61K 2800/34; A61Q 19/00; A61Q 17/04

(57) ABSTRACT

What are proposed are diesters of propane-1,3-diol with linear fatty acids having 6 to 22 carbon atoms and 0 or 1 to 3 double bonds, with the proviso that both the propane-1,3-diol and the fatty acids come exclusively from plant sources.

### FIELD OF THE INVENTION

The invention is in the field of cosmetics and relates to novel plant-based oil bodies having reduced tack, and to the use thereof as a substitute for silicone compounds.

### TECHNOLOGICAL BACKGROUND

For establishment of particular product properties, for example UV protection, moisture content, consistency and the like, cosmetic formulations comprise specific active ingredients and additives, for example UV filters, oil bodies or thickeners, but these, when applied to the skin, have the effect that the skin quickly feels dull, heavy and, in particular, sticky, and therefore such products are quickly considered to be inferior by the consumer.

The consumer desire for easily distributable, quickly spreading, non-heavy and non-tacky products is typically fulfilled by using silicone-based oils in particular. Mineral oil-based oils are also used as an alternative, but both groups are now considered to be problematic with regard to their biodegradability and the energy expenditure for production thereof. The limited availability thereof is likewise considered to be disadvantageous, since they originate from fossil sources.

### RELEVANT PRIOR ART

US 2009 0123398 A1 (PRESPERSE) recommends exchanging volatile silicone oils for volatile hydrocarbons. The aim is to establish the specific skin feel of cyclic silicones in particular in an alternative way.

US 2014 0356303 A1 (INOLEX) describes mixtures of polymeric esters and monomeric esters, likewise as a substitute for cyclic silicones.

EP 0914087 B1 (COGNIS) provides specific fat components that likewise find use as a substitute for silicone oils in haircare products; there is no mention of ester oils.

Finally, EP 1853219 A1 (COGNIS) describes the use of branched-chain oil bodies; there is no mention of linear structures.

Such biobased propane-1,3-diols are also proposed in EP 3215552 A1 (VANTAGE).

### OBJECT OF THE INVENTION

It was therefore an object of the present invention to provide alternative oil bodies that are suitable as a substitute for silicone compounds in general and cyclic silicones in particular, and at the same time in particular are easily distributable, spread quickly and do not feel either heavy or tacky on the skin. Moreover, it should be a priority that the substitutes are obtainable from renewable and especially from plant sources.

### DESCRIPTION OF THE INVENTION

The invention firstly provides diesters of propane-1,3-diol with linear fatty acids having 6 to 22 carbon atoms and 0 or 1 to 3 double bonds, with the proviso that both the propane-1,3-diol and the fatty acids come exclusively from plant sources.

It has been found that the diesters of the invention not only have an overall performance spectrum that fulfills the function; it was particularly surprising at the same time that the raw material basis specifically also for the propane-1,3-diol used has an influence on the sensory properties of the esterification product. More particularly, the propane-1,3-diol dicaprylate/caprate product which is particularly preferred in accordance with the invention has detectably lower tack when the diol component has been produced in a plant-based manner rather than in a petrochemical-based manner. It has not been possible to date to fully ascertain the cause of this, but it appears that particular by-products present in the petrochemical-based propane-1,3-diol adversely affect the performance properties thereof.

### Diesters

The diesters of the invention derive from propane-1,3-diol and corresponding linear, saturated or unsaturated fatty acids having 6 to 22 and especially 8 to 12 carbon atoms, and 0 and/or 1 to 3 double bonds. As usual in oleochemistry, what are used are generally not pure fatty acids but rather technical grade mixtures or fractions. Preference is given to using saturated fatty acids having 6 to 10 carbon atoms.

Suitable in principle are saturated fatty acids such as caproic acid, caprylic acid, capric acid, lauric acid, myristic acid, palmitic acid, stearic acid, arachidonic acid and behenic acid, and also the unsaturated fatty acids palmitoleic acid, oleic acid, elaidic acid, linoleic acid, linolenic acid, gadoleic acid and erucic acid. In addition, it is possible to use technical grade mixtures, such as palm oil fatty acid or coconut fatty acid. For all these fatty acids, it is a requirement that they are of plant origin, meaning that they are products that have been obtained by hydrolysis of oils such as palm oil, coconut oil, palm kernel oil, olive oil, sunflower oil and the like.

As elucidated above, it is critical that the propane-1,3-diol also comes from a plant source, whereas the customary preparation of propane-1,3-diol proceeds from petrochemical raw materials, for example from ethylene oxide, which is first hydroformylated. The aldehyde obtained as intermediate is then hydrogenated to give the diol. Corresponding products that are produced on the basis of corn, for example, are available on the market, for example, under the ZEMEA name from DuPont.

### Cosmetic Formulations

The invention further relates to cosmetic formulations comprising the diesters of the invention, preferably in amounts of about 0.1% to about 50% by weight, especially about 1% to about 30% by weight, more preferably about 2% to about 25% by weight and most preferably about 3% to about 15% by weight.

The formulations are preferably skincare products or suncare products that may be in the form of W/O or O/W emulsions. These formulations are preferably free of silicone compounds. “Free” in this connection should be understood to mean that the content of silicone compounds is below 1% by weight based on the formulations.

The compositions of the invention may comprise further typical auxiliaries and additives, for example mild surfactants, oil bodies, emulsifiers, pearlescent waxes, bodying agents, thickeners, superfatting agents, stabilizers, polymers, silicone compounds, fats, waxes, lecithins, phospholipids, UV light protection filters, pigments, humectants, biogenic active ingredients, antioxidants, deodorants, antiperspirants, antidandruff agents, film formers, swelling agents, insect repellents, self-tanning agents, tyrosine inhibitors (depigmenting agents), hydrotropes, solubilizers, preservatives, perfume oils, dyes and the like.

### Surfactants

Surface-active substances present may be anionic, nonionic, cationic and/or amphoteric or zwitterionic surfactants, the proportion of which in the compositions is typically about 1% to 70%, preferably 5% to 50% and especially 10% to 30% by weight.

Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, alkyl ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acyl amino acids, alkyl oligoglucoside sulfates, protein fatty acid condensates (especially wheat-based plant products) and alkyl (ether) phosphates. If the anionic surfactants contain polyglycol ether chains, these may have a conventional homolog distribution, but preferably have a narrowed homolog distribution. Particular preference is given in this connection to:
(a) acyl amino acid salts, for example acylglutamates, for example sodium acylglutamate, di-TEA palmitoyl aspartate and sodium caprylic/capric glutamate, acyl peptides, for example palmitoyl-hydrolyzed milk protein, sodium cocoyl-hydrolyzed soy protein and sodium/potassium cocoyl-hydrolyzed collagen and alaninates;
(b) acyl lactylates, lauroyl lactylate, caproyl lactylate
(c) sulfates, for example
   alkyl ether sulfates such as, in particular, sodium laureth sulfate, ammonium laureth sulfate, magnesium laureth sulfate, MIPA laureth sulfate, TIPA laureth sulfate, sodium myreth sulfate and sodium C12-13-pareth sulfate;
   alkyl sulfates, for example sodium lauryl sulfate, ammonium lauryl sulfate and TEA lauryl sulfate;
   glyceride sulfates, for example sodium cocomonoglyceride sulfate,
   amide sulfates, for example magnesium PEG-3-cocamide sulfate,
(d) sulfonates, for example
   alkyisulfonates,
   alkylarylsulfonates, especially sodium C12-14-olefinsulfonate,
(e) sulfosuccinates, for example dioctylsodium sulfosuccinate, disodium laureth sulfosuccinate, disodium lauryl sulfosuccinate and disodium undecylenamido-MEA sulfosuccinate;
(f) sulfoacetates, such as sodium lauryl sulfoacetate;
(g) sarcosinates, for example myristoylsarcosine, TEA lauroyl sarcosinate, sodium lauroylsarcosinate and sodium cocoylisarcosinate,
(l) isethionates, for example sodium/ammonium cocoylisethionate,
(h) taurates, for example sodium lauroyltaurate and sodium methylcocoyltaurate,
(i) carboxylates, for example
   soaps, for instance TEA stearate,
   ether carboxylates, for instance sodium laureth-13 carboxylate and sodium PEG-6 cocamide carboxylate,
(j) phosphates, for instance cetyl phosphate (mono-, dicetyl and mixtures thereof), potassium cetylphosphate, (mono-, dicetyl and mixtures thereof), DEA cetylphosphate (mono-, dicetyl and mixtures thereof), DEA oleth-10 phosphate and dilaureth-4 phosphate.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucuronic acid derivatives, fatty acid N-alkylglucamides, protein hydrolyzates (especially wheat-based plant products), polyol fatty acid esters, sugar esters, sorbitan esters, polysorbates and amine oxides. If the nonionic surfactants contain polyglycol ether chains, these may have a conventional homolog distribution, but preferably a narrowed homolog distribution.

Cationic surfactants contain at least one nitrogen atom covalently bonded to 4 alkyl or aryl groups. Irrespective of pH, this leads to a positive charge. Alkyl betaine, alkylamidopropyl betaine and alkylamidopropyl hydroxysultaines are advantageous. The cationic surfactants used may also preferably be selected from the group consisting of quaternary ammonium compounds, especially benzyltrialkylammonium chlorides or bromides, for example benzyldimethylstearylammonium chloride, and alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyridinium chloride, imidazoline derivatives and compounds having cationic character, such as amine oxides, for example alkyldimethylamine oxide or alkylaminoethyldimethylamine oxide. Cetyl trimethylammonium salts in particular are used advantageously. Particular preference is given here to:
alkylamines,
alkylimidazoles,
ethoxylated amines,
quaternary ammonium salts;
RNH₂CH₂CH₂COO⁻(at pH=7)
RNHCH₂CH₂COO⁻B⁺(at pH=12) B+=any desired cation, and
ester quats.

Typical examples of amphoteric or zwitterionic surfactants are alkyl betaines, alkylamido betaines, aminopropionates, aminoglycinates, imidazolinium betaines and sulfo betaines. The surfactants mentioned are exclusively known compounds.

Typical examples of particularly suitable mild, i.e. particularly skin-compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamido betaines, amphoacetals and/or protein fatty acid condensates, the latter preferably based on wheat proteins.

### Oil Bodies

As oil bodies come, for example, Guerbet alcohols based on fatty alcohols having 6 to 18, preferably 8 to 10 carbon atoms, esters of linear C₆-C₂₂ fatty acids with linear or branched C₆-C₂₂ fatty alcohols or esters of branched C₆-C₁₃ carboxylic acids with linear or branched C₆-C₂₂ fatty alcohols, for example myristyl myristate, myristyl palmitate, myristyl stearate, myristyl isostea rate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Particular preference is given to cetearyl ethylhexanoates, cetearyl nonanoate, stearyl heptanoate and stearyl caprylate, and mixtures thereof.

Additionally suitable are esters of linear C₆-C₂₂ fatty acids with branched alcohols, especially 2-ethylhexanol, esters of C₁₈-C₃₈-alkylhydroxycarboxylic acids with linear or branched C₆-C₂₂ fatty alcohols, especially dioctyl malates, esters of linear or branched C6-C13 carboxylic acids with linear or branched C6-C13 alcohols, for example ethylhexyl isononanoate, esters of linear and/or branched fatty acids with polyhydric alcohols (for example propylene glycol, dimer diol or trimer triol) and/or Guerbet alcohols, triglycerides based on C₆-C₁₀ fatty acids, liquid mono-/di-/triglyceride mixtures based on C₆-C₁₈ fatty acids, esters of C₆-C₂₂ fatty alcohols and/or Guerbet alcohols with aromatic carboxylic acids, especially benzoic acid, esters of C₂-C₁₂ dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂ fatty alcohol carbonates, for example Dicaprylyl Carbonate (Cetiol® CC), Guerbet carbonates based on fatty alcohols having 6 to 18, preferably 8 to 10, carbon atoms, esters of benzoic acid with linear and/or branched C₆-C₂₂ alcohols (e.g. Finsolv® TN), linear or branched, symmetric or unsymmetric dialkyl ethers having 6 to 22 carbon atoms per alkyl group, for example Dicaprylyl Ether (Cetiol® OE), ring-opening products of epoxidized fatty acid esters with polyols, silicone oils (cyclomethicones, silicomethicone types inter alia) and/or aliphatic or naphthenic hydrocarbons, for example such as squalane, squalene or dialkylcyclohexanes into consideration.

The amount used here, based on the final formulation, may be between 5% and 80% by weight, preferably between 10% and 50% by weight and especially between 20% and 40% by weight.

### Emulsifiers

Examples of useful emulsifiers include nonionogenic surfactants from at least one of the following groups:
addition products of 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and alkylamines having 8 to 22 carbon atoms in the alkyl radical;
alkyl and/or alkenyl oligoglycosides having 8 to 22 carbon atoms in the alk(en)yl radical and ethoxylated analogs thereof;
addition products of 1 to 15 mol of ethylene oxide onto castor oil and/or hydrogenated castor oil;
addition products of 15 to 60 mol of ethylene oxide onto castor oil and/or hydrogenated castor oil;
partial esters of glycerol and/or sorbitan with unsaturated linear or saturated branched fatty acids having 12 to 22 carbon atoms and/or hydroxycarboxylic acids having 3 to 18 carbon atoms and adducts thereof with 1 to 30 mol of ethylene oxide;
partial esters of polyglycerol (average self-condensation level of 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside) and polyglucosides (e.g. cellulose) with saturated and/or unsaturated, linear or branched fatty acids having 12 to 22 carbon atoms and/or hydroxycarboxylic acids having 3 to 18 carbon atoms and the adducts thereof with 1 to 30 mol of ethylene oxide;
mixed esters of pentaerythritol, fatty acids, citric acid and fatty alcohol and/or mixed esters of fatty acids having 6 to 22 carbon atoms, methyl glucose and polyols, preferably glycerol or polyglycerol;
wool wax alcohols;
polysiloxane-polyalkyl polyether copolymers or corresponding derivatives;
block copolymers, e.g. polyethylene glycol-30 dipolyhydroxystearate;
polymer emulsifiers, e.g. Pemulen grades (TR-1, TR-2) from Lubrizol or Cosmedia® SP from BASF;
polyalkylene glycols and
glycerol carbonate.

Particularly suitable emulsifiers are elucidated in detail hereinafter:

Alkoxylates. The addition products of ethylene oxide and/or of propylene oxide onto fatty alcohols, fatty acids, alkylphenols or castor oil are known, commercially available products. These are homologous mixtures having an average degree of alkoxylation corresponding to the ratio of the molar amounts of ethylene oxide and/or propylene oxide and substrate with which the addition reaction is performed. C_{12/18} fatty acid mono- and diesters of addition products of ethylene oxide onto glycerol are known as regreasing agents for cosmetic formulations.

Alkyl and/or alkenyl oligoglycoside. Alkyl and/or alkenyl oligoglycosides, the preparation thereof and the use thereof are known from the prior art. More particularly, they are prepared by reaction of glucose or oligosaccharides with primary alcohols having 8 to 18 carbon atoms. With regard to the glycoside residue, both monoglycosides in which a cyclic sugar radical is glycosidically bonded to the fatty alcohol and oligoglycosides having an oligomerization level of up to preferably about 8 are suitable. The oligomerization level is a statistical average based on a homolog distribution as is customary for such industrial products.

Partial glycerides. Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric diglyceride, malic acid monoglyceride, malic acid diglyceride, and technical grade mixtures thereof that may subordinately contain minor amounts of triglyceride from the production process. Likewise suitable are addition products of 1 to 30, preferably 5 to 10 mol, of ethylene oxide onto the partial glycerides mentioned.

Sorbitan esters. As sorbitan esters come sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical grade mixtures thereof. Likewise suitable are addition products of 1 to 30, preferably 5 to 10 mol, of ethylene oxide onto the sorbitan esters mentioned.

Polyglycerol esters. Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403) Polyglyceryl Dimerate Isostearate and mixtures thereof. Examples of further suitable polyol esters are the mono-, di- and triesters of trimethylolpropane or pentaerythritol with lauric acid, coconut fatty acid, tallow fatty acid, palmitic acid, stearic acid, oleic acid, behenic acid and the like, said mono-, di- and triesters optionally having been reacted with 1 to 30 mol of ethylene oxide.

Anionic emulsifiers. Typical anionic emulsifiers are aliphatic fatty acids having 12 to 22 carbon atoms, for example palmitic acid, stearic acid or behenic acid, and dicarboxylic acids having 12 to 22 carbon atoms, for example azelaic acid or sebacic acid.

Likewise suitable are mono-, di- and trialkyl phosphates and mono-, di- and/or tri-PEG-alkyl phosphates and salts thereof, for instance cetyl phosphate potassium salt, and the citrate esters, especially glyceryl oleate citrate and glyceryl stearyl citrate.

Amphoteric and cationic emulsifiers. Emulsifiers used may also be zwitterionic surfactants. Zwitterionic surfactants refer to those surface-active compounds that have, in the molecule, at least one quaternary ammonium group and at least one carboxylate group and one sulfonate group. Particularly suitable zwitterionic surfactants are the so-called betaines such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines each having 8 to 18 carbon atoms in the alkyl or acyl group, and cocoacylaminoethylhydroxyethylcarboxymethyl glycinate. Particular preference is given to the fatty acid amide derivative known by the INCI name Cocamidopropyl Betaine. Likewise suitable emulsifiers are ampholytic surfactants. Ampholytic surfactants are understood to mean those surface-active compounds that, apart from a C_{8/18}-alkyl or acyl group, contain at least one free amino group and at least one —COOH or —SO₃H group in the molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 carbon atoms in the alkyl group. Particularly preferred ampholytic surfactants are N-cocoalkylaminopropionate, cocoacylaminoethylaminopropionate and C_{12/18}-acylsarcosine. Finally, cationic surfactants are also useful as emulsifiers, with particular preference for those of the ester quat type, preferably methyl-quaternized di-fatty acid triethanolamine ester salts.

The amount of the emulsifiers used is typically within the range of about 0.5% to about 10% by weight and preferably about 1% to about 5% by weight.

### Fats and Waxes

Typical examples of fats are glycerides, i.e. solid or liquid, animal or vegetable products that consist essentially of mixed glycerol esters of higher fatty acids; useful waxes include natural or synthetic waxes, for example candelilla wax, carnauba wax, japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite, petrolatum, paraffin waxes, micro waxes; chemically modified waxes (hard waxes), for example montan ester waxes, sasol waxes, hydrogenated jojoba waxes and synthetic waxes, for example polyalkylene waxes and polyethylene glycol waxes. As well as the fats, useful additives also include fatlike substances such as lecithins and phospholipids. The term “lecithins” is understood by the person skilled in the art to mean those glycerophospholipids that form by esterification from fatty acids, glycerol, phosphoric acid and choline. Lecithins are therefore frequently also referred to in the specialist field as phosphatidylcholines (PC). Examples of natural lecithins include the cephalins, which are also referred to as phosphatidic acids and are derivatives of 1,2-diacyl-sn-glycerol-3-phosphoric acids. By contrast, phospholipids are usually understood to mean mono- and preferably diesters of phosphoric acid with glycerol (glycerol phosphates), which are generally counted among the fats. In addition, sphingosines or sphingolipids are also useful.

### Pearlescent Waxes

Examples of useful pearlescent waxes include: alkylene glycol esters, especially ethylene glycol distearate; fatty acid alkanolamides, especially coconut fatty acid diethanolamide; partial glycerides, especially stearic acid monoglyceride; esters of polyhydric, optionally hydroxy-substituted carboxylic acids with fatty alcohols having 6 to 22 carbon atoms, especially long-chain esters of tartaric acid; fatty substances, for example fatty alcohols, fatty ketones, fatty aldehydes, fatty ethers and fatty carbonates, which have a total of at least 24 carbon atoms, especially laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

### Physiological Cooling Substances

Cooling substances are compounds that generate a feeling of coldness on the skin. These are generally menthol compounds which—aside from the menthol parent itself—are selected, for example, from the group formed by Menthol Methyl Ether, Menthone Glyceryl Acetal (FEMA GRAS¹ 3807), Menthone Glyceryl Ketal (FEMA GRAS 3808), Menthyl Lactate (FEMA GRAS 3748), Menthol Ethylene Glycol Carbonate (FEMA GRAS 3805), Menthol Propylene Glycol Carbonate (FEMA GRAS 3806), menthyl N-ethyloxamate, Monomethyl Succinate (FEMA GRAS 3810), Monomenthyl Glutamate (FEMA GRAS 4006), menthoxy-1,2-propanediol ¹ FEMA stands, for “Flavor and Extracts Manufacturers Association” and GRAS is defined as “Generally Regarded As Safe”. A FEMA GRAS designation means that the substance thus characterized has been tested by standard methods and is considered to be toxicologically safe. (FEMA GRAS 3784), menthoxy-2-methyl-1,2-propanediol (FEMA GRAS 3849) and the menthanecarboxylic esters and -carboxamides WS-3, WS-4, WS-5, WS-12, WS-14 and WS-30, and mixtures thereof.

A first important representative of these substances is Monomenthyl Succinate (FEMA GRAS 3810). Both the succinate and the analogous Monomenthyl Glutarate (FEMA GRAS 4006) are important representatives of monomenthyl esters based on di- and polycarboxylic acids:

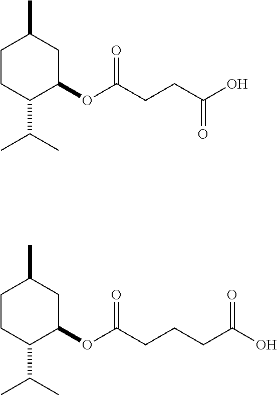 [SMILES: CC(C)[C@@H]1CC[C@@H](C)C[C@H]1OC(=O)CCC(=O)O ; CC(C)[C@@H]1CC[C@@H](C)C[C@H]1OC(=O)CCCC(=O)O]

Examples of applications of these substances can be found in documents WO 2003 043431 (Unilever) or EP 1332772 A1 (IFF), for example.

The next important group of menthol compounds that are preferred in the context of the invention includes carbonate esters of menthol and polyols, for example glycols, glycerol or carbohydrates, for example Menthol Ethyleneglycol Carbonate (FEMA GRAS 3805=Frescolat® MGC), Menthol Propyleneglycol Carbonate (FEMA GRAS 3784=Frescolat® MPC), Menthol 2-Methyl-1,2-propanediol Carbonate (FEMA GRAS 3849) or the corresponding sugar derivatives. Likewise preferred are the menthol compounds Menthyl Lactate (FEMA GRAS 3748=Frescolat® ML) and especially Menthone Glyceryl Acetal (FEMA GRAS 3807) or Menthone Glyceryl Ketal (FEMA GRAS 3808), which is marketed under the Frescolat® MGA name. Among these substances, those that have been found to be very particularly advantageous are Menthone Glyceryl Acetal/Ketal and Menthyl Lactate and Menthol Ethylene Glycol Carbonate or Menthol Propylene Glycol Carbonate, sold by the applicant under the Frescolat® MGA, Frescolat® ML, Frecolat® MGC and Frescolat® MPC names.

The 1970s saw the first development of menthol compounds having a C—C bond in the 3 position, a number of representatives of which can likewise be used. These substances are generally referred to as WS products. The parent is a menthol derivative in which the hydroxyl group has been replaced by a carboxyl group (WS-1). All other WS products derive from this structure, for example the preferred species WS-3, WS-4, WS-5, WS-12, WS-14 and WS-30.

### Bodying Agents and Thickeners

Useful bodying agents primarily include fatty alcohols or hydroxy fatty alcohols having 12 to 22 and preferably 16 to 18 carbon atoms, and additionally partial glycerides, fatty acids or hydroxy fatty acids. Preference is given to a combination of these substances with alkyl oligoglucosides and/or fatty acid N-methylglucamides of the same chain length and/or polyglyceryl poly-12-hydroxystearates. Suitable thickeners are, for example, Aerosil products (hydrophilic silicas), polysaccharides, especially xanthan gum, guar-guar, agar-agar, alginates and tyloses, carboxymethyl cellulose and hydroxyethyl and hydroxypropyl cellulose, and also higher molecular weight polyethylene glycol mono- and diesters of fatty acids, polyacrylates, (e.g. Carbopole® and Pemulen products from Lubrizol; Synthalene® from Sigma; Keltrol products from Kelco; Sepigel products from Seppic; Salcare products from BASF), polyacrylamides, polymers, polyvinyl alcohol and polyvinylpyrrolidone. Bentonites have also been found to be particularly effective, for example Bentone® Gel VS-5PC (Elementis), which is a mixture of cyclopentasiloxane, Disteardimonium Hectorite and propylene carbonate. Also useful surfactants, for example ethoxylated fatty acid glycerides, esters of fatty acids with polyols, for example pentaerythritol or trimethylolpropane, fatty alcohol ethoxylates having a narrowed homolog distribution or alkyl oligoglucosides, and electrolytes such as sodium chloride and ammonium chloride.

### Superfatting Agents and Stabilizers

Superfatting agents used may be substances such as, for example, lanolin and lecithin, and polyethoxylated or acylated lanolin and lecithin derivatives, polyol fatty acid esters, monoglycerides and fatty acid alkanolamides, where the latter serve simultaneously as foam stabilizers.

Stabilizers used may be metal salts of fatty acids, for example magnesium, aluminum and/or zinc stearate and/or magnesium, aluminum and/or zinc ricinoleate.

### Polymers

Suitable cationic polymers are, for example, cationic cellulose derivatives, for example a quaternized hydroxyethyl cellulose obtainable under the Polymer JR 400® name from Dow, cationic starch, copolymers of diallylammonium salts and acrylamides, quaternized vinylpyrrolidone/vinylimidazole polymers, for example Luviquat® (BASF), condensation products of polyglycols and amines, quaternized collagen polypeptides, for example Lauryldimonium Hydroxypropyl Hydrolyzed Collagen (Lamequat®L/BASF), quaternized wheat polypeptides, polyethyleneimine, cationic silicone polymers, for example amodimethicone, copolymers of adipic acid and dimethylaminohydroxypropyldiethylenetriamine (Cartaretine®/Sandoz), copolymers of acrylic acid with dimethyldiallylammonium chloride (Merquat® 550/Lubrizol), polyaminopolyamides and the crosslinked water-soluble polymers thereof, cationic chitin derivatives, for example quaternized chitosan, optionally in microcrystalline distribution, condensation products of dihaloalkyls, for example dibromobutane, with bisdialkylamines, for example bis(dimethylamino-1,3-propane), cationic guar gum, for example Jaguar® CBS, Jaguar® C-17, Jaguar® C-16 (Solvay), quaternized ammonium salt polymers, for example Mirapol® A-15, Mirapol® AD-1, Mirapol® AZ-1 (Solvay).

Examples of useful anionic, zwitterionic, amphoteric and nonionic polymers include vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and those crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl nnethacrylate/vinylcaprolactam terpolymers, and optionally derivatized cellulose ethers and silicones.

### Silicone Compounds

Suitable silicone compounds are, for example, dimethylpolysiloxanes, methylphenylpolysiloxanes, cyclic silicones, and amino-, fatty acid-, alcohol-, polyether-, epoxy-, fluorine-, glycoside- and/or alkyl-modified silicone compounds that may be either in liquid or resin form at room temperature. Also suitable are simethicones, which are mixtures of dimethicones having an average chain length of 200 to 300 dimethylsiloxane units and hydrogenated silicates.

### Uv Light Protection Filters

It has been found that the diesters of the invention are especially capable of overcoming the tack that is typical of many UV filters. The present invention therefore further relates to formulations which, as well as the diesters, further comprise at least one UV filter. Especially preferred are those formulations comprising
(a) propane-1,3-diol dicaprylate/caprate and
(b) at least one UV light protection filter,
   with the proviso that component (a) has been produced entirely in a plant-based manner. Likewise typical are formulations comprising
(a) about 0.1% to about 30% by weight of propane-1,3-diol dicaprylate/caprate and
(b) about 1% to about 50% by weight of UV light protection filter,
   with the proviso that the stated amounts together with solvents and further cosmetic auxiliaries and additives add up to 100% by weight.

UV light protection filters (also often referred to synonymously as light protection factors) are, for example, organic substances that are liquid or in crystalline form at room temperature and are capable of absorbing ultraviolet rays and releasing the energy absorbed again in the form of long-wave radiation, for example heat. The UV light protection filters are typically present in amounts of 0.1% to 50% and preferably 1% to 45% by weight.

Useful typical UV-A filters especially include derivatives of benzoylmethane, for example 1-(4′-tert-butylphenyl)-3-(4′-methoxyphenyl)propane-1,3-dione, 4-tert-butyl-4′-methoxydibenzoylmethane (Neo Heliopan® 357), 2-(4-diethylamino-2-hydroxybenzoyl)-benzoic acid hexyl ester (Uvinul® A Plus), 1-phenyl-3-(4′-isopropylphenyl)propane-1,3-dione and enamine compounds. Particular preference is given to:
terephthalylidenedibornanesulfonic acid and salts (Mexoryl® SX);
hexyl 2-(4-diethylamino-2-hydroxybenzoyl)benzoate (Uvinul® A Plus);
2,2′-(1,4-phenylene)bis[1H-benzimidazole-4,5-disulfonic acid], disodium salt (Neo Heliopan® AP);
menthyl anthranilate (Neo Heliopan® MA);
   and mixtures thereof.

UVB filters may be oil-soluble or water-soluble. Examples of oil-soluble substances include:
octocrylene;
homosalate;
octisalate;
p-aminobenzoic acid;
ethyl p-aminobenzoate+25E0;
2-ethylhexyl p-dimethylaminobenzoate;
triethanolamine salicylate (Neo Heliopan® TS);
menthyl anthranilate (Neo Heliopan® MA);
2-ethylhexyl p-methoxycinnamate (Neo Heliopan® AV);
isoamyl p-methoxycinnamate (Neo Heliopan®E 1000);
2-phenylbenzimidazolesulfonic acid (Neo Heliopan® Hydro) and salts thereof;
3-(4′-trimethylammonium)benzylidenebornan-2-one methylsulfate;
3-(4′-sulfo)benzylidenebornan-2-one and salts thereof;
3-(4′-methylbenzylidene)-d,l-camphor (Neo Heliopan® MBC);
N-[(2,4)-[2-(oxoborn-3-ylidene)methyl]benzyl]acrylamide polymer;
4,4′-[(6-[4-(1,1-dimethyl)aminocarbonyl)phenylamino]-1,3,5-triazine-2,4-diyl) diimino] bis(benzoic acid 2-ethylhexyl ester) (Uvasorb® HEB);
benzylidenemalonate-polysiloxane (Parsol® SLX);
tris(2-ethylhexyl) 4,4′,4″-(1,3,5-triazine-2,4,6-triyltriimino)tribenzoate (Uvinul® T150);
methoxy propylamino cyclohexenylidene ethoxyethyl cyanoacetate
   and mixtures thereof.

Suitable broadband filters include, for example:
2-hydroxy-4-methoxybenzophenone-5-sulfonic acid (sulisobenzone, benzophenone-4)
   and salts thereof;
2-hydroxy-4-methoxybenzophenone (Neo Heliopan® BB);
disodium 2,2′-dihydroxy-4,4′-dimethoxy-5,5′-disulfobenzophenone;
phenol, -(2H-benzotriazol-2-yl-4-methyl-6-(2-methyl-3-(1,3,3,3-tetramethyl-1-(trimethylsilyl)oxy)disiloxyanyl)propyl), (Mexoryl® XL);
2,2′-methylenebis(6-(2H-benztriazol-2-yl)-4-1,1,3,3-tetra methylbutyl)phenol (Tinosorb® M);
Tris-Biphenyl Triazine (Tinosorb® A2B);
bemotrizinol (Neo Heliopan® BMT);
2,4-bis[[(4-(3-sulfonato)-2-hydroxypropyloxy)-2-hydroxy]phenyl]-6-(4-methoxyphenyl)-1,3,5-triazine sodium salt;
2,4-bis[[(3-(2-propyloxy)-2-hydroxypropyloxy)-2-hydroxy]phenyl]-6-(4-methoxyphenyl)-1,3,5-triazine;
2,4-bis[[4-(2-ethylhexyloxy)-2-hydroxy]phenyl]-6-[4-(2-methoxyethylcarbonyl)phenylamino]-1,3,5-triazine;
2,4-bis[[4-(3-(2-propyloxy)-2-hydroxypropyloxy)-2-hydroxy]phenyl]-6[4-(2-ethyl carboxyl)phenylamino]-1,3,5-triazine;
2,4-bis[[4-(2-ethylhexyloxy)-2-hydroxy]phenyl]-6-(1-methylpyrrol-2-yl)-1,3,5-triazine;
2,4-bis[[4-tris(trimethylsiloxysilylpropyloxy)-2-hydroxy]phenyl]-6-(4-methoxy phenyl)-1,3,5-triazine;
2,4-bis[[4-(2″-methylpropenyloxy)-2-hydroxy]phenyl]-6-(4-methoxyphenyl)-1,3,5-triazine;
2,4-bis[[4-(1′,1′,1′,3′,5′,5′,5′-heptamethylsiloxy-2″-methylpropyloxy)-2 hydroxy]phenyl]-6-(4-methoxyphenyl)-1,3,5-triazine;
(5,6, 5′,6′-tetraphenyl-3,3′-(1,4-phenylene)bis(1,2,4-triazine) and mixtures thereof.

The UV-A and UV-B filters may of course also be used in mixtures. Particularly favorable combinations consist of the derivatives of benzoylmethane, e.g. 4-tert-butyl-4′-methoxydibenzoylmethane (Neo Heliopan® 357) and 2-cyano-3,3-phenylcinnamic acid 2-ethylhexyl ester (octocrylene) in combination with esters of cinnamic acid, preferably 2-ethylhexyl 4-methoxycinnamate and/or propyl 4-methoxycinnamate and/or isoamyl 4-methoxycinnamate. Advantageously, such combinations are combined with water-soluble filters, for example 2-phenylbenzimidazole-5-sulfonic acid and the alkali metal, alkaline earth metal, ammonium, alkylammonium, alkanolammonium and glucammonium salts thereof.

### Pigments, Especially Light Protection Pigments

As well as the soluble substances mentioned, insoluble light protection pigments are also useful for this purpose, namely finely dispersed metal oxides or salts. Examples of suitable metal oxides are especially zinc oxide and titanium dioxide, and additionally oxides of iron, zirconium, silicon, manganese, aluminum and cerium, and mixtures thereof. Salts used may be silicates (talc), barium sulfate or zinc stearate. The oxides and salts are used in the form of the pigments for skincare and skin protection emulsions and decorative cosmetics. The particles here should have an average diameter of less than 100 nm, preferably between 5 and 50 nm and especially between 15 and 30 nm. They may have a spherical shape, but it is also possible to use those particles which have an ellipsoidal shape or shape that varies from the spherical form in some other way. The pigments may also have been surface-treated, i.e. may be in hydrophilized or hydrophobized form. Typical examples are coated titanium dioxides, for example T805 titanium dioxide (Degussa) or Eusolex® T2000, Eusolex® T, Eusolex® T-ECO, Eusolex® T-S, Eusolex® T-Aqua, Eusolex® T-45D (all Merck), Uvinul TiO₂ (BASF). Useful hydrophobic coating agents here include silicones in particular, and specifically trialkoxyoctylsilanes or simethicones. In sunscreens, preference is given to using what are called micro- or nanopigments. Preference is given to using micronized zinc oxide, for example Z-COTE® or Z-COTE HP1®.

### Humectants

Humectants serve to further optimize the sensory properties of the composition and to regulate skin moisture. At the same time, the cold stability of the formulations of the invention, especially in the case of emulsions, is increased. The humectants are typically present in an amount of 0.1% to 15% by weight, preferably 1% to 10% by weight, and especially 5% to 10% by weight.

The following are among those suitable in accordance with the invention: amino acids, pyrrolidonecarboxylic acid, lactic acid and salts thereof, lactitol, urea and urea derivatives, uric acid, glucosamine, creatinine, cleavage products of collagen, chitosan or chitosan salts/derivatives, and especially polyols and polyol derivatives (e.g. glycerol, diglycerol, triglycerol, ethylene glycol, propylene glycol, butylene glycol, erythritol, hexane-1,2,6-triol, polyethylene glycols such as PEG-4, PEG-6, PEG-7, PEG-8, PEG-9, PEG-10, PEG-12, PEG-14, PEG-16, PEG-18, PEG-20), sugars and sugar derivatives (including fructose, glucose, maltose, maltitol, mannitol, inositol, sorbitol, sorbitylsilanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolyzates and mixtures of hydrogenated wheat protein and PEG-20 acetate copolymer. Humectants suitable with preference in accordance with the invention are glycerol, diglycerol, triglycerol and butylene glycol.

### Biogenic Active Ingredients and Antioxidants

Biogenic active ingredients are understood to mean, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example Prunus extract, bambara nut extract and vitamin complexes.

Antioxidants interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, acetophenone derivatives, such as 4-hydroxyacetophenone in particular, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

### Deodorants and Antimicrobial Agents

Cosmetic deodorants counteract, mask or eliminate body odor. Body odor results from the action of skin bacteria on apocrine perspiration, forming unpleasant-smelling degradation products. Accordingly, deodorants contain active ingredients that function as antimicrobial agents, enzyme inhibitors, odor absorbers or odor maskers.

Antimicrobial agents. Suitable antimicrobial agents are in principle all substances that are active against Gram-positive bacteria, for example 2-methyl-5-cyclohexylpentanol, 1,2-decylene glycol, 4-hydroxybenzoic acid and its salts and esters, N-(4-chlorophenyl)-N′-(3,4-dichlorphenyl)urea, 2,4,4′-trichloro-2′-hydroxydiphenyl ether (triclosan), 4-chloro-3,5-dimethylphenol, 2,2′-methylenbis(6-bromo-4-chlorophenol), 3-methyl-4-(1-methylethyl)-phenol, 2-benzyl-4-chlorphenol, 3-(4-chlorophenoxy)propane-1,2-diol, 3-iodo-2-propynyl butylcarbamate, chlorhexidine, 3,4,4′-trichlorocarbanilide (TTC), antibacterial odorants, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), N-alkylsalicylamides, for example n-octylsalicylamide or n-decylsalicylamide.

Enzyme inhibitors. Examples of suitable enzyme inhibitors include esterase inhibitors. These are preferably trialkyl citrates such as trimethyl citrate, tripropyl citrate, triisopropyl citrate, tributyl citrate and especially triethyl citrate (Hydagen® CAT). The substances inhibit enzymatic activity and hence reduce odor formation. Further substances useful as esterase inhibitors are sterol sulfates or phosphates, for example lanosterol sulfate or phosphate, cholesterol sulfate or phosphate, campesterol sulfate or phosphate, stigmasterol sulfate or phosphate and sitosterol sulfate or phosphate, dicarboxylic acids and esters thereof, for example glutaric acid, monoethyl glutarate, diethyl glutarate, adipic acid, monoethyl adipate, diethyl adipate, malonic acid and diethyl malonate, hydroxycarboxylic acids and esters thereof, for example citric acid, malic acid, tartaric acid or diethyl tartrate, and zinc glycinate.

Odor absorbers. Suitable odor absorbers are substances that can absorb and largely retain odor-forming compounds. They lower the partial pressure of the individual components and hence also reduce the speed at which they spread. It is important that they do not affect perfumes. Odor absorbers have zero efficacy against bacteria. They contain, for example, as a main constituent, a complex zinc salt of ricinoleic acid or specific, largely odor-neutral fragrances that are known to the person skilled in the art as “fixatives”, for example extracts of labdanum or styrax or particular abietic acid derivatives. The function of odor maskers is fulfilled by odorants or perfume oils which, in addition to their function as odor maskers, impart their respective fragrance note to deodorants. Examples of useful perfume oils include mixtures of natural and synthetic odorants. Natural odorants are extracts from blossoms, stems and leaves, fruits, fruit peel, roots, woods, herbs and grasses, needles and branches, and resins and balsams. Also useful are animal raw materials, for example cibet and castoreum. Typical synthetic odorant compounds are products of the ester, ether, aldehyde, ketone, alcohol and hydrocarbon type. Odorant compounds of the ester type are, for example, benzyl acetate, p-tert-butylcyclohexyl acetate, linalyl acetate, phenylethyl acetate, linalyl benzoate, benzyl formate, allylcyclohexyl propionate, styrallyl propionate and benzyl salicylate. The ethers include, for example, benzyl ethyl ether; the aldehydes include, for example, linear alkanals having 8 to 18 carbon atoms, citral, citronellal, citronellyloxyacetaldehyde, cyclamen aldehyde, hydroxycitronellal, lilial and bourgeonal; the ketones include, for example, the ionones and methyl cedryl ketone; the alcohols include anethole, citronellol, eugenol, isoeugenol, geraniol, linalool, phenylethyl alcohol and terpineol; the hydrocarbons include mainly the terpenes and balsams. However, preference is given to using mixtures of different odorants that together produce a pleasing fragrance note. Also suitable as perfume oils are essential oils of lower volatility that are usually used as aroma components, for example sage oil, chamomile oil, clove oil, melissa oil, mint oil, cinnamon leaf oil, linden blossom oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, Evernyl, Iraldein gamma, phenylacetic acid, geranyl acetate, benzyl acetate, rose oxide, romilat, irotyl and floramat, alone or in mixtures.

Antiperspirants. Antiperspirants reduce perspiration by influencing the activity of the eccrine perspiration glands, and hence counteract armpit wetness and body odor. Aqueous or anhydrous formulations of antiperspirants typically contain the following ingredients:
astringent active ingredients,
oil components,
nonionic emulsifiers,
coemulsifiers,
bodying agents,
auxiliaries, for example thickeners or complexing agents, and/or
nonaqueous solvents, for example ethanol, propylene glycol and/or glycerol.

Suitable astringent antiperspirant active ingredients are, in particular, salts of aluminum, of zirconium or of zinc. Such suitable antihidrotic active ingredients are, for example, aluminum chloride, aluminum chlorohydrate, aluminum dichlorohydrate, aluminum sesquichlorohydrate and complexes thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complexes thereof, for example with amino acids such as glycine. In addition, antiperspirants may include customary oil-soluble and water-soluble auxiliaries in relatively small amounts. Such oil-soluble auxiliaries may be, for example:
inflammation-inhibiting, skin-protecting or pleasant-smelling essential oils,
synthetic skin-protecting active ingredients and/or
oil-soluble perfume oils.

Customary water-soluble additives are, for example, preservatives, water-soluble fragrances, pH modifiers, e.g. buffer mixtures, water-soluble thickeners, e.g. water-soluble natural or synthetic polymers, for example xanthan gum, hydroxyethyl cellulose, polyvinylpyrrolidone or high molecular weight polyethylene oxides.

### Film Formers

Commonly used film formers are, for example, chitosan, microcrystalline chitosan, quaternized chitosan, polyvinylpyrrolidone, vinylpyrrolidone-vinyl acetate copolymers, polymers from the acrylic acid series, quaternary cellulose derivatives, hydrolyzed jojoba esters, collagen, hyaluronic acid or salts thereof, and similar compounds.

### Anti Dandruff Active Ingredients

Useful antidandruff active ingredients include piroctone olamine (1-hydroxy-4-methyl-6-(2,4,4-trimethylpentyl)-2-(1H)-pyridinone monoethanolamine salt), Crinipan® AD (climbazole), Ketoconazol®, (4-acetyl-1-{-4[-2-(2,4-dichlorophenyl) r-2-(1H-imidazol-1-ylmethyl)-1,3-dioxylan-c-4-ylmethoxyphenyl}piperazine, ketoconazole, elubiol, selenium disulfide, colloidal sulfur, sulfur polyethylene glycol sorbitan monooleate, sulfur ricinol polyethoxylate, sulfur tar distillates, salicylic acid (or in combination with hexachlorophene), undecylenoic acid monoethanolamide sulfosuccinate Na salt, Lamepon® UD (protein-undecylenoic acid condensate), zinc pyrithione, aluminum pyrithione and magnesium pyrithione/dipyrithione-magnesium sulfate.

### Swelling Agents

Swelling agents used for aqueous phases may be montmorillonites, clay minerals, pemulene and alkyl-modified Carbopol grades (Lubrizol). Other suitable polymers or swelling agents can be found in the review by R. Lochhead in Cosm.Toil. 108, 95 (1993).

### Insect Repellents

Useful insect repellents include N,N-diethyl-m-toluamide, pentane-1,2-diol or ethyl butylacetylaminopropionates. A suitable self-tanning agent is dihydroxyacetone. Useful tyrosine inhibitors that prevent the formation of melanin and find use in depigmenting agents include, for example, arbutin, ferulic acid, kojic acid, coumarin acid and ascorbic acid (vitamin C).

### Ingredients for Oral and Dental Care Compositions

Toothpastes or tooth creams are generally understood to mean formulations in gel or paste form that are composed of water, thickeners, humectants, abrasive or cleaning bodies, surfactants, sweeteners, aromas, deodorizing active ingredients, and active ingredients to counter oral and dental disorders. It is possible to use any customary cleaning bodies in the toothpastes of the invention, for example chalk, dicalcium phosphate, insoluble sodium metaphosphate, aluminum silicates, calcium pyrophosphate, finely divided synthetic resins, silicas, alumina and alumina trihydrate.

Preferentially suitable cleaning bodies for the toothpastes of the invention are, in particular, finely divided xerogel silicas, hydrogel silicas, precipitated silicas, alumina trihydrate and finely divided alpha-alumina or mixtures of these cleaning bodies in amounts of 15% to 40% by weight of the toothpaste. Useful humectants include predominantly low molecular weight polyethylene glycols, glycerol, sorbitol or mixtures of these products in amounts of up to 50% by weight. Suitable examples among the known thickeners are the thickening finely divided gel silicas and hydrocolloids, for example carboxymethyl cellulose, hydroxyethyl cellulose, hydroxypropyl guar, hydroxyethyl starch, polyvinylpyrrolidone, high molecular weight polyethylene glycol, plant gums such as tragacanth, agar-agar, carrageenan moss, gum arabic, xanthan gum and carboxyvinyl polymers (e.g. Carbopol® grades). In addition to the mixtures of menthofuran and menthol compounds, the oral and dental care compositions may especially comprise surface-active substances, preferably anionic and nonionic high-foaming surfactants, such as the substances already mentioned above, but especially alkyl ether sulfate salts, alkyl polyglucosides and mixtures thereof.

Further customary toothpaste additives are:
preservatives and antimicrobial substances, for example methyl, ethyl or propyl p-hydroxybenzoate, sodium sorbate, sodium benzoate, bromochlorophene, phenyl salicylate, thymol and the like;
antitartar active ingredients, for example organophosphates such as 1-hydroxyethane-1,1-diphosphonic acid, 1-phosphonpropane-1,2,3-tricarboxylic acid and others that are known, for example, from U.S. Pat. No. 3,488,419, DE 2224430 A1 and DE 2343196 A1;
other caries-inhibiting substances, for example sodium fluoride, sodium monofluorophosphate, tin fluoride;
sweeteners, for example saccharin-sodium, sodium cyclamate, sucrose, lactose, maltose, fructose or Apartam®, (L-aspartyl-L-phenylalanine methyl ester), Stevia extracts or the sweetening constituents thereof, especially ribeaudioside;
additional aromas, for example eucalyptus oil, aniseed oil, fennel oil, caraway oil, methyl acetate, cinnamaldehyde, anethole, vanillin, thymol and mixtures of these and other natural and synthetic aromas;
pigments, for example titanium dioxide;
dyes;
buffer substances, for example primary, secondary or tertiary alkali metal phosphates or citric acid/sodium citrate;
wound-healing and inflammation-inhibiting substances, for example allantoin, urea, azulene, chamomile active ingredients and acetylsalicylic acid derivatives.

A preferred execution of the cosmetic formulations is toothpastes in the form of an aqueous pasty dispersion comprising polishing agents, humectants, viscosity regulators and optionally further customary components, and the mixture of menthofuran and menthol compounds in amounts of 0.5% to 2% by weight.

In mouthwashes, a combination with aqueous-alcoholic solutions having different levels of essential oils, emulsifiers, astringent and tonifying drug extracts, antitartar, antibacterial additives and odor correctors is directly possible. A further preferred execution of the invention is a mouthwash in the form of an aqueous or aqueous-alcoholic solution containing the mixture of menthofuran and menthol compounds in amounts of 0.5% to 2% by weight. In mouthwashes that are diluted prior to use, it is possible to achieve sufficient effects with higher concentrations in accordance with the envisaged dilution ratio.

### Hydrotropes

For improvement of flow characteristics, it is also possible to use hydrotropes, for example ethanol, isopropyl alcohol or polyols; these substances correspond largely to the carriers outlined at the outset. Polyols that are an option here preferably have 2 to 15 carbon atoms and at least two hydroxyl groups. The polyols may also contain further functional groups, especially amino groups, or have been modified with nitrogen. Typical examples are
glycerol;
alkylene glycols, for example ethylene glycol, diethylene glycol, propylene glycol, butylene glycol, pentylene glycol, caprylyl glycol and polyethylene glycols having an average molecular weight of 100 to 1000 daltons;
technical oligoglycerol mixtures having a self-condensation level of 1.5 to 10, for instance technical diglycerol mixtures having a diglycerol content of 40% to 50% by weight;
methylol compounds such as, in particular, trimethylolethane, trimethylolpropane, trimethylolbutane, pentaerythritol and dipentaerythritol;
lower alkyl glucosides, especially those having 1 to 8 carbons in the alkyl radical, for example methyl and butyl glucoside;
sugar alcohols having 5 to 12 carbon atoms, for example sorbitol or mannitol,
sugars having 5 to 12 carbon atoms, for example glucose or sucrose;
amino sugars, for example glucamine;
dialcoholamines, such as diethanolamine or 2-aminopropane-1,3-diol.

### Preservatives

Suitable preservatives are, for example, phenoxyethanol, formaldehyde solution, parabens, o-cymen-5-ol, tropolone or sorbic acid, also the silver complexes known by the Surfacine® name and the further substance classes listed in Annex 6, Parts A and B of the Cosmetics Regulation.

### Perfume Oils and Aromas

Perfume oils include mixtures of natural and synthetic odorants. Natural odorants are extracts from flowers (lily, lavender, rose, jasmine, neroli, ylang-ylang), stems and leaves (geranium, patchouli, petitgrain), fruits (aniseed, coriander, cumin, juniper), fruit peel (bergamot, lemon, orange), roots (mace, angelica, celery, cardamon, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also useful are animal raw materials, for example cibet and castoreum. Typical synthetic odorant compounds are products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allylcyclohexyl propionate, styrallyl propionate and benzyl salicylate. The ethers include, for example, benzyl ethyl ether; the aldehydes include, for example, linear alkanals having 8 to 18 carbon atoms, citral, citronellal, citronellyloxyacetaldehyde, cyclamen aldehyde, hydroxycitronellal, lilial and bourgeonal; the ketones include, for example, the ionones, α-isomethylionone and methyl cedryl ketone; the alcohols include anethole, citronellol, eugenol, isoeugenol, geraniol, linalool, phenylethyl alcohol and terpineol; the hydrocarbons include mainly the terpenes and balsams. However, preference is given to using mixtures of different odorants that together produce a pleasing fragrance note. Also suitable as perfume oils are essential oils of lower volatility that are usually used as aroma components, for example sage oil, chamomile oil, clove oil, melissa oil, mint oil, cinnamon leaf oil, linden blossom oil, juniperberry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix Coeur, Iso-E-Super, Fixolide NP, Evernyl, Iraldein gamma, phenylacetic acid, geranyl acetate, benzyl acetate, rose oxide, romilat, irotyl and floramat, alone or in mixtures.

Examples of useful aromas include peppermint oil, spearmint oil, aniseed oil, star anise oil, caraway oil, eucalyptus oil, fennel oil, lemon oil, wintergreen oil, clove oil, menthol and the like.

### Dyes

Dyes used may be substances that are approved and suitable for cosmetic purposes, as compiled, for example, in the publication “Kosmetische Färbemittel” [Cosmetic Dyes] from the Dyes Commission of the German Research Foundation, Verlag Chemie, Weinheim, 1984, p. 81-106. Examples are cochineal red A (C.I. 16255), patent blue V (C.I.42051), indigotin (C.I.73015), chlorophyllin (C.I.75810), quinoline yellow (C.I.47005), titanium dioxide (C.I.77891), indanthrene blue RS (C.I. 69800) and madder (C.I.58000). Luminol may also be present as a luminescent dye. These dyes are typically used in concentrations of 0.001% to 0.1% by weight, based on the overall mixture.

### Formulations

Preferred compositions of the invention are selected from the group of products for treatment, for protection, for care and for cleaning of the skin and/or the hair, or as a makeup product, either as leave-on or rinse-off products.

The formulations include, for example, dispersions, suspensions, creams, lotions or milk, according to the production method and ingredients, gels (including hydrogels, e.g. hydrodispersion gels, oleogels), sprays (e.g. pump sprays or sprays with propellant), foams or impregnation solutions for cosmetic tissues, soaps, washing liquids, shower and bath preparations, bath products (capsules, oil, tablets, salts, bath salts, soaps etc.), effervescent preparations, skincare products, for example emulsions, ointments, pastes, gels (as described above), oils, balsams, sera, powders (e.g. face powders, body powders), masks, pencils, roll-on sticks, aerosols (foaming, non-foaming or after-foaming), deodorants and/or antiperspirants, mouthwashes and mouth rinses, insect repellents, sunscreens, aftersun preparations, shaving products, aftershave balsams, pre- and aftershave lotions, hair removal products, haircare products, for example shampoos (including 2-in-1 shampoos, antidandruff shampoos, baby shampoos, shampoos for a dry scalp, concentrated shampoos), conditioners, hair tonics, hair lotions, hair rinses, styling cremes, hair greases, permanent wave and setting lotions, hair sprays, e.g. styling aids (e.g. gel or wax), hair straightening agents (disentangling agents, relaxing agents), hair dyes, for example temporary hair dyes, semipermanent hair dyes, permanent hair dyes, hair conditioners, hair foams, eye care products, makeups, makeup removers or baby products.

More preferably, the formulations of the invention take the form of an emulsion, especially the form of a W/O, O/W, W/O/W or O/W/O emulsion, PIT emulsion, B. a Pickering emulsion, an emulsion having a low oil content, a micro- or nanoemulsion, a gel (including hydrogel, hydrodispersion gel, oleogel) or a solution.

The total content of the auxiliaries and additives may be 1% to 50%, preferably 5% to 40% by weight—based on the compositions. The compositions can be produced by customary low- or high-temperature processes; or else by the phase inversion temperature method.

### INDUSTRIAL APPLICABILITY

The invention further relates to the use of the diesters of the invention as oil components, and especially as a substitute for silicone compounds in cosmetic formulations.

Likewise claimed is a method of improving the sensory properties of a cosmetic formulation, comprising or consisting of the following steps:
(a) providing a cosmetic formulation having a content of silicone compounds and
b) exchanging the silicone compounds for the diesters as claimed in claim 1.

The cosmetic formulations are preferably sunscreens containing at least one UV filter.

It should be pointed out that preferred embodiments mentioned in association with the diesters of the invention and the formulations containing them, especially combinations and stated ranges, are also applicable to the uses and methods of the invention, without requiring any repetition.

### EXAMPLES

### Example 1, Comparative Examples V1 to V3

Various skincare cremes were produced using 5% by weight each of a silicone oil, plant-based short-chain lipids, and one petrochemical-based and one plant-based propane-1,3-diol dicaprylate/caprate, and then, after 2 g of the cremes had been rubbed onto the back of the hand, subjected to sensory assessment by a panel of five experienced testers.

.

The average values from the assessments are shown in Table 1. For the two parameters examined, (1)=not very marked, (2)=distinct and (3)=very distinct.

TABLE 1
Sensory assessment

| Trade name | INCI name | V1 | V2 | V3 | 1 |
|---|---|---|---|---|---|
| Emulgade | Cetearyl Glucoside (and) Ceteareth | 5.0 | 5.0 | 5.0 | 5.0 |
| PL6850 | Alcohol | | | | |
| Amphisol K | Potassium Cetyl Phosphate | 0.50 | 0.50 | 0.50 | 0.50 |
| Cutina GMS-V | Glyceryl Stearate | 1.00 | 1.00 | 1.00 | 1.00 |
| Wacker AK350 | Dimethicone | 5.0 | — | — | — |
| silicone oil | | | | | |
| Myritol 318 | Caprylic/Capric Triglyceride | — | 5.0 | — | — |
| | 1,3-Propanediol Dicaprylate/caprate | — | — | 5.0 | — |
| | 1,3-Propanediol Dicaprylate/caprate | — | — | — | 5.0 |
| | (Bio) | | | | |
| Novata AB | Cocoglycerides | 1.0 | 1.0 | 1.0 | 1.0 |
| Carbopol 980 | Carbomer | 0.3 | 0.3 | 0.3 | 0.3 |
| | Glycerine | 0.3 | 0.3 | 0.3 | 0.3 |
| | Aqua | Ad 100 | | | |
| Sensory assessment | | | | | |
| Spreading capacity | | 2.0 | 2.0 | 2.5 | 3.0 |
| Tackiness | | 3.0 | 2.5 | 1.5 | 1.0 |

The creme of the invention is notable for higher spreading capacity and distinctly lower tackiness compared to all comparative products.

1. A diester of propane-1,3-diol with a linear fatty acid having 6 to 22 carbon atoms and 0 or 1 to 3 double bonds, with the proviso that both the propane-1,3-diol and the fatty acid come exclusively from plant sources.

2. A diester according to claim 1, wherein the fatty acid is saturated.

3. A diester according to claim 1, wherein the fatty acid is caprylic acid, capric acid, or mixtures thereof.

4. A cosmetic formulation comprising one or more diesters of claim 1.

5. The cosmetic formulation as claimed in claim 4, comprising the one or more diesters in an amount of about 0.1% to about 50% by weight.

6. The cosmetic formulation, according to claim 4, wherein the cosmetic formulation is a skincare product or a suncare product.

7. The cosmetic formulation, according to claim 4, wherein the cosmetic formulation is in the form of a W/O or O/W emulsion.

8. The cosmetic formulation according to claim 4, further comprising at least one UV filter.

9. The cosmetic formulation according to claim 4, further comprising:
(a) propane-1,3-diol dicaprylate/caprate; and
(b) at least one UV light protection filter,
with the proviso that component (a) has been produced entirely in a plant-based manner.

10. The cosmetic formulation according to claim 9, wherein the cosmetic formulation comprises:
(a) about 0.1% to about 30% by weight of propane-1,3-diol dicaprylate/caprate; and
(b) about 1% to about 50% by weight of UV light protection filter,
with the proviso that the stated amounts together with solvents and further cosmetic auxiliaries and additives add up to 100% by weight.

11. The cosmetic formulation, according to claim 4, wherein the cosmetic formulation is free of silicone compounds.

12. A method comprising using one or more diesters according to claim 1 as oil components.

13. A method comprising using one or more diesters according to claim 1 as a substitute for silicone compounds in cosmetic formulations.

14. A method of improving the sensory properties of a cosmetic formulation, comprising:
(a) providing a cosmetic formulation having a content of silicone compounds and
b) exchanging the silicone compounds for one or more diesters according to claim 1.

15. The method, according to claim 14, wherein the cosmetic formulation comprises at least one UV filter.
